(19) United States
(12) Patent Application Publication
(10) Pub. No.: US 2011/0038929 A1
(43) Pub. Date: Feb. 17, 2011

(54) ORAL CARE PET PRODUCT AND METHOD

(76) Inventor: Susan D. Weiss, Bonita Springs, FL (US)

Correspondence Address: Brinks Hofer Gilson & Lione/Ann Arbor, 524 South Main Street, Suite 200, Ann Arbor, MI, 48104, US

(21) Appl. No.: 12/867,527

(22) PCT Filed: Feb. 13, 2009
(86) PCT No.: PCT/US09/34033

§ 371 Date: Nov. 3, 2010

Related U.S. Application Data
(60) Provisional application No. 61/028,414, filed on Feb. 13, 2008.

(51) Int. Cl. A61K 8/66; A61K 9/14; A61K 9/46; A61Q 11/00

(52) U.S. Cl. 424/466; 424/44; 424/499

(57) ABSTRACT

An effervescent composition for reducing breath odors and preventing and/or reducing plaque buildup, deposits and/or formation on the teeth of pet animals. The effervescent composition includes at least one active agent adapted to reduce or destroy bacteria present in the mouth of the pet animal responsible for causing bad breath and plaque; and an effervescent component including a carbonate containing compound and an acidifying compound.

The present application claims priority to U.S. provisional application entitled ORAL CARE PET PRODUCT AND METHOD having Ser. No. 61/028,414 and filed on Feb. 13, 2008.

### FIELD OF THE INVENTION

The present invention generally relates to products for oral pet care and methods of providing oral pet care. More particularly, the invention relates to a plaque and bad breath preventative and to methods of preventing bad breath and plaque and tarter build-up in pets, namely dogs and cats.

### BACKGROUND OF THE INVENTION

Pets, such as dogs and cats, are susceptible to a variety of dental health issues. Some of these issues include bad breath, plaque and tarter formation, gum irritation and stained teeth. The source of many of these problems originates in the chemistry of the pet's mouth, and in particular, in its saliva.

Plaque is a sticky film, consisting of bacteria and other components, that becomes very tightly adhered to the teeth. Once adhered, it is not easily washed away. Tartar, also known as dental calculus, results from the thickening and hardening (mineralization) of plaque. As tartar develops, it becomes visibly white or yellowish in color and may become stained by foods or other products eaten or chewed by the pet.

While being undesirable from a purely aesthetic standpoint, tartar build-ups are also a source of irritation in the pet's mouth and are a contributing factor in gingivitis. Once formed, tartar is virtually impossible to remove, except by mechanical measures, the most common form of which is abrasively scraping the tartar off of the pet's teeth.

Plaque bacteria are also believed to produce acids that eat away the enamel of the teeth. This in turn results in tooth decay of and cavities. Tooth decay begins at the surface of the tooth and may eventually progress through the dentine of the tooth and into the pulp cavity. Once in the pulp cavity, this decay may result in loss of the tooth.

The bacteria in a pet's mouth are also a significant cause of bad breath. A wide variety of chemical and biological agents have been suggested to slow or prevent plaque and tarter formation or to remove plaque after it has formed. In humans, prevention and removal of this material is routinely done by brushing, flossing, using mouth washes and by mechanical removal (scraping and polishing) at a dentist's office. When it comes to pets, however, the pet owner, while being well intentioned, usually only briefly adheres to a routine of dental hygiene for their pets. Compounding this, many pets find brushing and flossing by their owners to be an unpleasant experience and, therefore, are uncooperative during the process.

Since pet foods do not provide sufficient cleaning to remove plaque (pet food is typically consumed very rapidly with little chewing on the part of the pet), a wide variety of chew products have been manufactured for pets. These chew products are intended to provide a mechanically abrasive action that will remove the plaque and tartar, while also massaging the gums of the pet. Such products include pieces of rawhide (in various forms and shapes), rope (knotted on its ends to resemble bones and other structures), as well as biscuits and treats having a formulation that requires additional chewing on the part of the pet and thereby results in additional abrasive action to remove the plaque. In addition, each of these products may be provided with chemical or biological agents that also operate to prevent plaque build up or bad breath.

With the present invention, an alternative product and method for reducing or alleviating the above dental problems is provided.

### SUMMARY

In overcoming the above drawbacks and limitations of the know technology, the present invention provides an effervescent composition for reducing breath odors and preventing and/or reducing plaque buildup, deposits and/or formation on the teeth of pet animals, the effervescent composition comprising: at least one active agent adapted to reduce or destroy bacteria present in the mouth of the pet animal responsible for causing bad breath and plaque; and an effervescent component including a carbonate containing compound and an acidifying compound.

In another aspect of the invention, the carbonate containing compound is baking soda.

In a further aspect of the invention, the acidifying compound is citric acid

In yet another aspect of the invention, the effervescent compound is provided in powder form.

In still a further aspect of the invention, the effervescent compound is provided in tablet form.

In still another aspect of the invention, the at least one active agent is a naturally occurring enzyme.

In another aspect of the invention, the naturally occurring enzyme is at least one naturally occurring enzyme selected from the group of lysozyme, glucoxidase, lactoferrin, amylase, amyloglucosidase, peptizyme and papain.

In yet another aspect of the invention, the at least one active agent is a naturally occurring enzyme operating to inhibit bacterial growth.

In a further aspect of the invention, the at least one active agent is a naturally occurring enzyme operating to attack and destroy bacteria.

In still another aspect of the invention, the effervescent composition further includes an analgesic.

In a further aspect of the invention, the effervescent composition includes a colorant.

In another aspect of the invention, the effervescent composition includes an odor masking component.

In yet another aspect of the invention, the effervescent composition includes a taste enhancing compound.

In still another aspect of the invention, the effervescent composition includes an anti-inflammatory compound.

In a further aspect of the invention, the effervescent composition includes a preservative.

In still a further aspect of the invention, the effervescent composition is dissolved in water as a carrier medium.

In another aspect of the invention, a method of reducing breath odors and preventing and/or reducing plaque buildup, deposits and/or formation on the teeth of pet animals is provided, the method comprising the steps of: providing an effervescent composition having one or more active agents adapted to reduce or destroy bacteria present in the mouth of the pet and responsible for causing bad breath and plaque; treating one of food or drinking water of the pet by dispensing the effervescent composition onto or into the food or drinking water; and providing the treated food or drinking water to the pet for consumption by the pet.

In another aspect of the invention, the step of providing the effervescent composition includes providing the one or more active agents as naturally occurring enzymes adapted to inhibit bacterial growth or to attack and destroy bacteria.

In another aspect of the invention, the step of providing the effervescent composition includes selecting the one or more active agents from the group of lysozyme, glucoxidase, lactoferrin, amylase, amyloglucosidase, peptizyme and papain.

### BRIEF DESCRIPTION OF THE DRAWINGS

The drawing is a diagrammatic illustration of the pet dental hygiene product incorporating the principles of the present invention, in two different forms, being applied to drinking water in a pet's bowl.

### DETAILED DESCRIPTION

In overcoming the drawbacks and limitations as outlined above and elsewhere, the present invention provides a pet dental hygiene product that operates on the bacteria in a pet's mouth to reduce and/or prevent the development of plaque, tartar, and the problems associated therewith. Additionally, the product also reduces or eliminates bad breath of a pet that is caused by bacteria.

The product is an effervescent composition 10 that includes a one or more active agents that, by way of changing the chemistry of the pet's saliva, reduce or eliminate the bacteria that causes the development of plaque and bad breath. The active agents are preferably a variety of naturally occurring enzymes, but could also be other chemical or biological agents that would achieve the same or similar effects.

To dispense the product to the pet, the effervescent composition 10 is preferably emptied into water 12 in the pet's water bowl 14. Upon contact with the water, the effervescent constituents, a carbonate containing compound and an acidifying compound, react with one another producing a rapid evolution of carbon dioxide gas. The carbon dioxide gas operates to stir the contents of the water bowl, dispersing and solubilizing the active agents. Once dispersed in the pet's drinking water, the product is preferably odorless, colorless and tasteless. In the water, the active agents remain bioavailable and effective for a period of time that depends on the particular active agents. Thus, every time the pet drinks treated water from the water bowl a dosage of active agents is applied to the pet's teeth and mouth, destroying the plaque and bad breath producing bacteria. By delivering the dental product via this method, the daily chore of brushing the pet's teeth (including the application of cleansing wipes, sprays, gels, and pastes), which the pet may not enjoy and may not permit without some resistance, is eliminated. With the present invention, a dose of the composition 10 in every fresh bowl of drinking water, preferably at least daily, is all that is needed for better pet dental health.

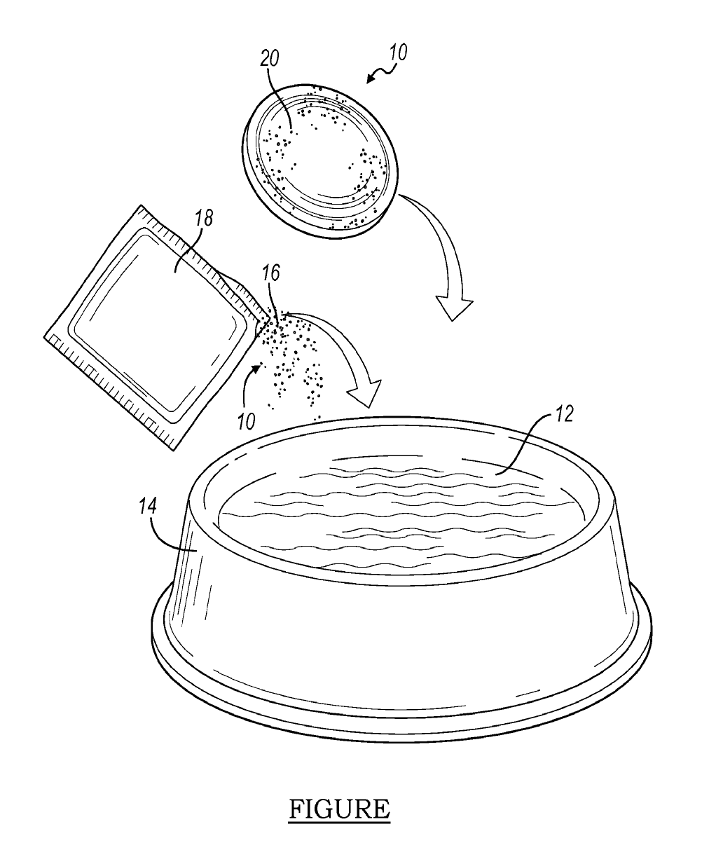

In a preferred embodiment of the invention, as seen in FIG. 1a, the effervescent composition 10 is provided in powder form 16. As such, a single dosage of the effervescent composition 10 is preferably carried in a packet 18, which may be torn open so that its contents can be poured into the pet's water bowl 14. However, the composition 10 could be provided alternately in a tablet form 20 As with the powder form 16, the tablet form 20 may be similarly packaged in a packet 18 that can be torn open so that the tablet may be dispensed into the pet's water bowl.

As noted above, the effervescent components of the composition 10 includes a carbonate containing compound and an acidifying compound. One preferred carbonate containing compound is baking soda, although other carbonate compounds may be used. A preferred acidifying compound is citric acid. Obviously, other acidifying compounds may similarly be used.

For the active agents, it is preferred that a variety of all natural constituents are used. Thus, the preferred active agents are naturally occurring enzymes that function to destroy the plaque and bad breath causing bacteria present in the pet's mouth and saliva. Preferably, a combination of active agents is used to provide multiple mechanisms by which bacterial growth and plaque are inhibited and reduced.

In one preferred embodiment, the active agent component is a blend of naturally occurring enzymes including, as it principle constituents, lysozyme, glucoxidase, lactoferrin, amylase, amyloglucosidase, peptizyme and papain. A blend of these specific enzymes is available from Specialty Enzymes and Biochemicals Co. of Signal Hill, Calif. under the brand name Bactase™.

Lysozyme is a protein that attacks the cell walls of certain bacteria. By attacking the cell wall, lysozyme weakens the cell walls and eventually the cell wills burst, destroying the bacteria.

Glucoxidase is a microbial enzyme that reacts with oxygen. By reacting with oxygen, this enzyme operates to deprive aerobic bacteria of the oxygen they need for survival.

Lactoferrin is an iron binding glucoprotein and bacteriostat. Not intending to be bound by theory, it is believed that by binding with iron, this protein destroys bacteria through iron deprivation.

Amylases are enzymes that break down starches into maltose molecules. Enzymes of this type are major digestive enzymes.

Amyloglucosidase is a dextrinizing enzyme and breaks down complex foods containing sugars. Since sugars cause cavities and promote bacteria growth, reducing sugars in the pet's mouth will aid in reducing both of these and the problems associated therewith.

Peptizyme is effective in controlling infections and is also an anti-inflammatory enzyme. As such, its presence helps control dental infections and also helps in reducing plaque.

Papain is a protease. This proteas helps whiten teeth and control plaque.

While the preferred embodiment includes the above constituents, it is contemplated that the invention could employ various ones of these specific or similar constituents, either individually or in other combinations with one another. Thus, the invention is not intended to be construed as expressly limited to only the above combination of active agents. Also, other active agents providing similar benefits could be employed as alternative for the specifically identified active agents.

If desired, additional ingredients may be added to the composition 10 so as to affect various other attributes of composition 10, such as shelf life, taste, formability, admixing, analgesic properties, etc. Such ingredients may include one or more of the following: sodium benzoate, sodium salicylate, sodium lauryl sulfate, sodium borate, sodium citrate, chlorhexidine gluconate, potassium sorbate, xylitol, sorbate of potassium, zinc, zinc acetate, zinc gluconate, anthium dioxide, stabilized chlorine dioxide, hydrogen peroxide, chlorine peroxide, stabilized chlorine dioxide, lactoperoxidase, dextranase, sodium hydroxide, and yucca schidigera.

In an alternative embodiment, an odor masking component may be added to the effervescent composition. By employing an odor masking component, non-bacterial derived breath odor, such as food source breath odor, may be controlled.

Another alternative embodiment may incorporate a colorant in the effervescent composition. The colorant would provide a colored tint to the pet's drinking water, thereby indicating to the pet owner that the dental preventative of the invention had already been dispensed into the drinking water. This would be useful in situations where multiple people take care of a common pet.

In another aspect of the invention, the dental preventative may be provided to the pet without dispensing the preventative in the pet's drinking water. Cats, for example, are not typically large water drinkers. For such pets, the effervescent composition of the present invention may be applied to the pet's food, such as sprinkling the effervescent composition on top of or mixing into their food. If the food is moist, at least some of the effervescent components will react, but dispersing of the active agents throughout the food will not be accomplished.

In another alternative form, the composition 10 is provided in a capsule form that will dissolve in the pet's drinking water or food. As a further alternate form, the composition 10 could be within a pet treat, biscuit or similar product.

It is anticipated that the composition of the present invention could be provided in yet other forms, including as a powder in bulk form (requiring individual dispensing by the pet owner); as a gel or paste that is added to the pet's drinking water; or as a bottled liquid product where the ingredients of the composition 10 have been already dissolved in water as a carrier medium such that all or some of the contents of the bottle are emptied into the pet's drinking bowl, already with or without water.

As a person skilled in the art will readily appreciate, the above description is meant as an illustration of implementation of the principles this invention. This description is not intended to limit the scope or application of this invention in that the invention is susceptible to modification, variation and change, without departing from spirit of this invention, as defined in the following claims.

1. An effervescent composition for reducing breath odors and preventing and/or reducing plaque buildup, deposits and/or formation on the teeth of pet animals, the effervescent composition comprising:
a blend of active agents adapted to reduce or destroy bacteria present in the mouth of the pet animal responsible for causing bad breath and plaque, wherein the blend of active agents is a blend of naturally occurring enzymes selected from the group of lysozyme, glucoxidase, lactoferrin, amylase, amyloglucosidase, peptizyme and papain, and wherein at least one of the naturally occurring enzymes operates to inhibit bacterial growth and at least one other of the naturally occurring enzymes operates to attack and destroy bacteria; and
an effervescent component including a carbonate containing compound and an acidifying compound.

2. The effervescent composition as set out in claim 1 wherein the carbonate containing compound is baking soda.

3. The effervescent composition as set out in claim 1 wherein the acidifying compound is citric acid.

4. The effervescent composition as set out in claim 1 wherein the effervescent compound is provided in powder form.

5. The effervescent composition as set out in claim 1 wherein the effervescent compound is provided in tablet form.

6-9. (canceled)

10. The effervescent composition as set out in claim 1 further comprising an analgesic.

11. The effervescent composition as set out in claim 1 further comprising a colorant.

12. The effervescent composition as set out in claim 1 further comprising an odor masking component.

13. The effervescent composition as set out in claim 1 further comprising a taste enhancing compound.

14. The effervescent composition as set out in claim 1 further comprising an anti-inflammatory compound.

15. The effervescent composition as set out in claim 1 further comprising a preservative.

16. The effervescent composition as set out in claim 1 dissolved in water as a carrier medium.

17. A method of reducing breath odors and preventing and/or reducing plaque buildup, deposits and/or formation on the teeth of pet animals, the method comprising the steps of:
providing an effervescent composition having a blend of active agents adapted to reduce or destroy bacteria present in the mouth of the pet and responsible for causing bad breath and plaque wherein the blend of active agents are provided as a blend of naturally occurring enzymes selected from the group of lysozyme, glucoxidase, lactoferrin, amylase, amyloglucosidase, peptizyme and papain, at least one of the enzymes is adapted to inhibit bacterial growth and at least one other of the enzymes is adapted to attack and destroy bacteria;
treating one of food or drinking water of the pet by dispensing the effervescent composition onto or into the food or drinking water; and
providing the treated food or drinking water to the pet for consumption by the pet.

18-19. (canceled)
